(19) United States
(12) Patent Application Publication
(10) Pub. No.: US 2020/0138845 A1
(43) Pub. Date: May 7, 2020

(54) Combination Drug Therapy

(71) Applicant: ViiV Healthcare Company, Wilmington, DE (US)

(72) Inventor: Trevor R. SCOTT, Research Triangle Park, NC (US)

(21) Appl. No.: 16/631,014

(22) PCT Filed: Jul. 16, 2018
(86) PCT No.: PCT/IB2018/055257

§ 371 Date: Jan. 14, 2020

Related U.S. Application Data
(60) Provisional application No. 62/533,696, filed on Jul. 18, 2017.

(51) Int. Cl. A61K 31/7076; A61K 31/4985

(52) U.S. Cl. CPC A61K 31/7076; A61K 31/4985

(57) ABSTRACT

A novel combination comprising the integrase strand transfer inhibitor, cabotegravir or a pharmaceutically acceptable salt or solvate thereof, with the nucleoside reverse transcriptase translocation inhibitor EFdA (MK-8591), or a pharmaceutically acceptable salt or solvate thereof, pharmaceutical compositions comprising the same and methods of using such combinations and compositions in the treatment of conditions in which the inhibition of the HIV integrase or reverse transcriptase is beneficial, e.g., HIV.

### FIELD OF THE INVENTION

The present invention relates to a method of treating HIV and to combinations useful in such treatment. In particular, the method relates to a novel combination comprising the integrase strand transfer inhibitor, cabotegravir or a pharmaceutically acceptable salt or solvate thereof, with the nucleoside reverse transcriptase translocation inhibitor (NRTTI), 4′-ethynyl-2-fluoro-2′-deoxyadenosine, known as EFdA (MK-8591), or a pharmaceutically acceptable salt or solvate thereof, pharmaceutical compositions comprising the same and methods of using such combinations and compositions in the treatment of conditions in which the inhibition of the HIV integrase or reverse transcriptase is beneficial, e.g., HIV.

### BACKGROUND OF THE INVENTION

Over the past decades, advances in highly-active antiretroviral therapies (ARTs) have improved treatment efficacy for patients with HIV, improving patient survival and quality of life. However, proper adherence to treatment regimens remains a challenge where poor compliance can result in treatment failure and the emergence of drug-resistant mutations. To help aid adherence, longer acting treatments are under investigation. Both oral and long-acting injectable ART may provide patients with a convenient and discreet approach to manage HIV infection.

Cabotegravir (GSK1265744) is an integrase strand transfer inhibitor (INSTI) that exhibits subnanomolar potency and antiviral activity against a broad range of HIV-1 strains. Oral administration of cabotegravir has exhibited acceptable safety and tolerability profiles, a long half-life, and few drug-drug interactions. In the phase IIb LAT trial (ClinicalTrials.gov identifier, NCT01641809), a two-drug regimen of once-daily oral formulations of cabotegravir and rilpivirine demonstrated durable viral suppression in previously suppressed subjects, providing proof of principle for a two-drug maintenance regimen using Cabotegravir and an non-nucleoside reverse transcriptase inhibitors (NNRTI).

Long-acting injectable formulations of cabotegravir are also in clinical development. Clinical studies investigating long-acting cabotegravir have demonstrated prolonged exposures (≥30 days) following injection, enabling dosing at once-monthly or longer intervals. A long-acting combination of cabotegravir and rilpivirine have proven clinically successful at long term treatment of HIV (Margolis D A, Podzamczer D, Stellbrink H-J, et al. Cabotegravir+Rilpivirine as Long-Acting Maintenance Therapy: LATTE-2 Week 48 Results. 21st International AIDS Conference, Durban, South Africa. Jul. 18-22, 2016. Abstract THAB0206LB).

Although current combinations of cabotegravir have proven successful, it is desired to provide an even more beneficial combination of cabotegravir with a second HIV-treating agent.

### SUMMARY OF THE INVENTION

The present inventors have identified a particular combination of HIV inhibiting agents that provide improved properties over either agent alone or prior combinations of otherwise similar agents. In particular, the combination comprising the integrase strand transfer inhibitor (INSTI), cabotegravir or a pharmaceutically acceptable salt or solvate thereof, with the nucleoside reverse transcriptase translocation inhibitor (NRTTI), 4′-ethynyl-2-fluoro-2′-deoxyadenosine, known as EFdA (MK-8591), or a pharmaceutically acceptable salt or solvate thereof, pharmaceutical compositions comprising the same and methods of using such combinations and compositions in the treatment of conditions in which the inhibition of the HIV integrase or reverse transcriptase is beneficial, e.g., HIV, are described herein.

The INSTI inhibitor of the invention is represented by the structure of formula (I):

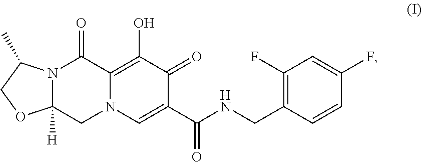 [SMILES: C[C@H]1CO[C@@H]2Cn3cc(C(=O)NCc4ccc(F)cc4F)c(=O)c(O)c3C(=O)N12]

or a pharmaceutically acceptable salt or solvate thereof (collectively referred to herein as “compound A”, the free base form of which is referred to as cabotegravir),

The NRTTI inhibitor of the invention is represented by the structure of formula (II):

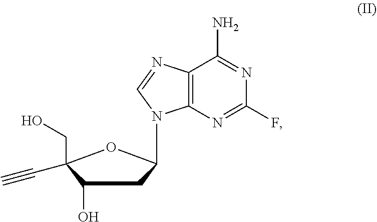 [SMILES: C#C[C@]1(CO)O[C@@H](n2cnc3c(N)nc(F)nc32)C[C@H]1O]

or a pharmaceutically acceptable salt or solvate thereof (collectively referred to herein as “compound B”, the free form of which is known as MK-8591 or EFdA),

In a first aspect of the present invention, there is provided a combination comprising:

(i) a compound of formula (I)

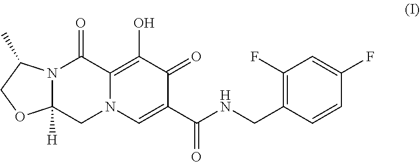 [SMILES: C[C@H]1CO[C@@H]2Cn3cc(C(=O)NCc4ccc(F)cc4F)c(=O)c(O)c3C(=O)N12]

or a pharmaceutically acceptable salt or solvate thereof and
(ii) a compound of formula (II)

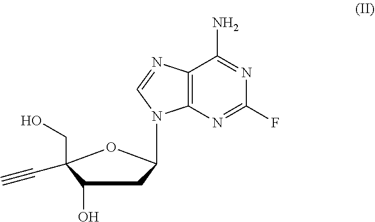 [SMILES: C#C[C@]1(CO)O[C@@H](n2cnc3c(N)nc(F)nc32)C[C@H]1O]

or a pharmaceutically acceptable salt or solvate thereof.

In one aspect of the invention, the INSTI compound of formula I is in a salt form. In a preferred embodiment, the salt form of the INSTI compound is in the sodium salt.

In another aspect of the present invention, there is provided a combination comprising:

(i) a compound of formula (I):

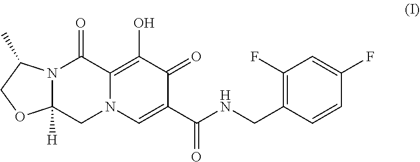 [SMILES: C[C@H]1CO[C@@H]2Cn3cc(C(=O)NCc4ccc(F)cc4F)c(=O)c(O)c3C(=O)N12]

or a pharmaceutically acceptable salt or solvate thereof and
(ii) a compound of formula (II):

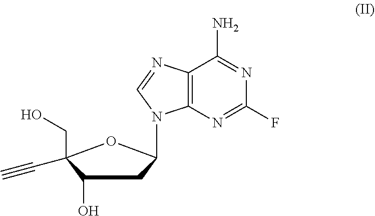 [SMILES: C#C[C@]1(CO)O[C@@H](n2cnc3c(N)nc(F)nc32)C[C@H]1O]

or a pharmaceutically acceptable salt or solvate thereof for use in therapy.

In another aspect of the present invention, there is provided a combination comprising:

(i) a compound of formula (I):

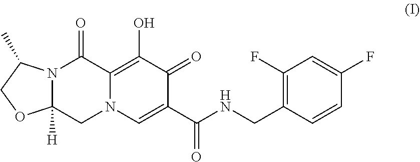 [SMILES: C[C@H]1CO[C@@H]2Cn3cc(C(=O)NCc4ccc(F)cc4F)c(=O)c(O)c3C(=O)N12]

or a pharmaceutically acceptable salt or solvate thereof and
(ii) a compound of formula (II):

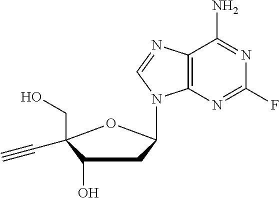 [SMILES: C#C[C@]1(CO)O[C@@H](n2cnc3c(N)nc(F)nc32)C[C@H]1O]

or a pharmaceutically acceptable salt or solvate thereof, for use in treatment of HIV.

In another aspect of the present invention, there is provided a combination comprising:

(i) a compound of formula (I):

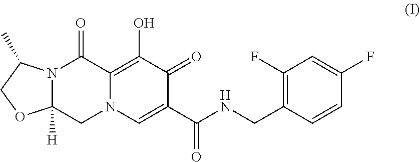 [SMILES: C[C@H]1CO[C@@H]2Cn3cc(C(=O)NCc4ccc(F)cc4F)c(=O)c(O)c3C(=O)N12]

or a pharmaceutically acceptable salt or solvate thereof and
(ii) a compound of formula (II):

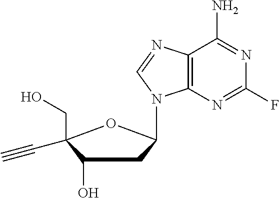 [SMILES: C#C[C@]1(CO)O[C@@H](n2cnc3c(N)nc(F)nc32)C[C@H]1O]

or a pharmaceutically acceptable salt or solvate thereof, together with a pharmaceutically acceptable diluent or carrier.

In another aspect of the present invention, there is provided a combination comprising:

(i) a compound of formula (I):

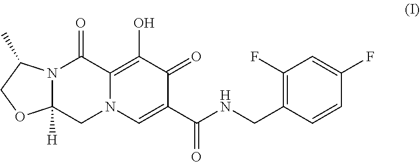 [SMILES: C[C@H]1CO[C@@H]2Cn3cc(C(=O)NCc4ccc(F)cc4F)c(=O)c(O)c3C(=O)N12]

or a pharmaceutically acceptable salt or solvate thereof and
(ii) a compound of formula (II):

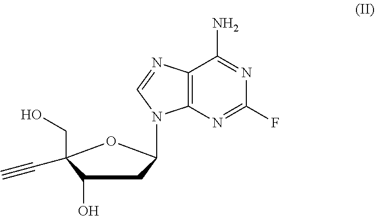 [SMILES: C#C[C@]1(CO)O[C@@H](n2cnc3c(N)nc(F)nc32)C[C@H]1O]

or a pharmaceutically acceptable salt or solvate thereof in the manufacture of a medicament for the treatment of HIV.

In another aspect of the present invention, there is provided a method of treatment of HIV in a mammal comprising administering to said mammal:

a therapeutically effective amount of a compound of formula (I)

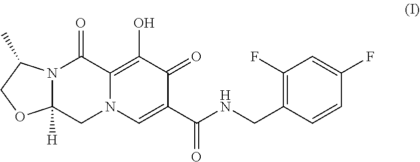 [SMILES: C[C@H]1CO[C@@H]2Cn3cc(C(=O)NCc4ccc(F)cc4F)c(=O)c(O)c3C(=O)N12]

or a pharmaceutically acceptable salt or solvate thereof and
(ii) a compound of formula (II):

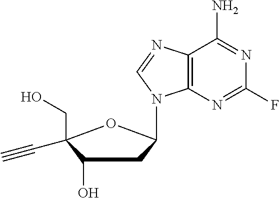 [SMILES: C#C[C@]1(CO)O[C@@H](n2cnc3c(N)nc(F)nc32)C[C@H]1O]

or a pharmaceutically acceptable salt or solvate thereof.

In a further aspect of this invention is provided a method of treating HIV in a human in need thereof which comprises administering a therapeutically effective amount of a combination of the invention wherein the combination is administered within a specific period, and for a duration of time.

### DETAILED DESCRIPTION

### Definitions

As used herein, the term “co-administer” refers to administration of two or more agents within a 24-hour period of each other, for example, as part of a clinical treatment regimen. In other embodiments, “co-administer” refers to administration of two or more agents within 2 hours of each other. In other embodiments, “co-administer” refers to administration of two or more agents within 30 minutes of each other. In other embodiments, “co-administer” refers to administration of two or more agents within 15 minutes of each other. In other embodiments, “co-administer” refers to administration at the same time, either as part of a single formulation or as multiple formulations that are administered by the same or different routes.

“Cabotegravir” is the compound defined by the structure

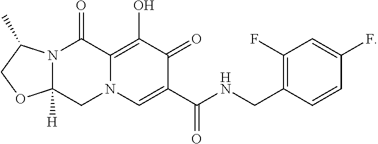 [SMILES: C[C@H]1CO[C@@H]2Cn3cc(C(=O)NCc4ccc(F)cc4F)c(=O)c(O)c3C(=O)N12]

Cabotegravir may be associated with sodium. Methods for preparing cabotegravir are described in U.S. Pat. 8,410,103. Cabotegravir has been demonstrated to be efficacious in treatment and prevention of HIV both in oral and parenteral dosage forms. See for instance, Margolis D A, Brinson C C, Eron J J, et al. 744 and Rilpivirine as Two Drug Oral Maintenance Therapy: LAI116482 (LATTE) Week 48 Results. 21st Conference on Retroviruses and Opportunistic Infections (CROI); Mar. 3-6, 2014; Boston, Mass., Margolis D A, Podzamczer D, Stellbrink H-J, et al. Cabotegravir+Rilpivirine as Long-Acting Maintenance Therapy: LATTE-2 Week 48 Results. 21st International AIDS Conference; Jul. 18-22, 2016; Durban, South Africa, Abstract THAB0206LB. Levin: Conference reports for National AIDS Treatment Advocacy Project (NATAP); 2016, and Markowitz M, Frank I, Grant R, et al. ECLAIR: Phase 2A Safety and PK Study of Cabotegravir LA in HIV-Uninfected Men. Abstract presented at: 23rd Conference on Retroviruses and Opportunistic Infections (CROI); Feb. 22-25, 2016; Boston, Mass.

In an alternative embodiment of the invention, the Cabotegravir component may be formulated as a pro-drug of the base compound, such pro-drug being optionally a salt as described herein. Particular pro-drugs of cabotegravir include, for instance, international patent publications WO2010/011814 and WO2010/011815

“EFdA is the compound defined by the structure:

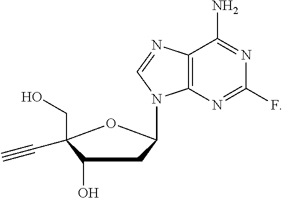 [SMILES: C#C[C@]1(CO)O[C@@H](n2cnc3c(N)nc(F)nc32)C[C@H]1O]

EFdA is described in U.S. Pat. No. 7,339,053. EFdA has been studied in clinical studies for the treatment of HIV, and is also known as MK-8591, or 4′-ethynyl-2-fluoro-2′-deoxyadenosine. Upon administration to a patient, EFdA is converted to EFdA-5′-triphosphate (EFdATP) which binds to the polymerase active site of HIV-1 reverse transcriptase.

“Therapeutically effective amount” or “effective amount” refers to that amount of the compound being administered that will prevent a condition, or will relieve to some extent one or more of the symptoms of the disorder being treated. Pharmaceutical compositions suitable for use herein include compositions wherein the active ingredients are contained in an amount sufficient to achieve the intended purpose. Determination of a therapeutically effective amount is well within the capability of those skilled in the art, especially in light of the detailed disclosure provided herein.

As used herein, the term “treatment” or “treating” in the context of therapeutic methods, refers to alleviating the specified condition, eliminating or reducing the symptoms of the condition, slowing or eliminating the progression, invasion, or spread of the condition and reducing or delaying the reoccurrence of the condition in a previously afflicted subject. The present invention further provides use of the compounds of the invention for the preparation of a medicament for the treatment of several conditions in a mammal (e.g., human) in need thereof.

As used herein, the term “prevention” or “preventing” in the context of therapeutic methods, refers to precluding the specified condition or symptoms of the condition, or in the occurrence of prior infection, precluding the re-occurance of the condition. The present invention further provides use of the compounds of the invention for the preparation of a medicament for the prevention of several conditions in a mammal (e.g., human) in need thereof.

As referenced, the dosage forms herein may be used to treat or, alternatively, prevent HIV which unless further clarified is intended to mean HIV-1. As an alternative embodiment, the combination of the invention may also be effective against HIV-2, or against patients having dual HIV-1/HIV-2 infection.

### Dosage Form

In certain embodiments, Compound A and Compound B, a r e in a unitary dosage form for simultaneous administration to a patient, for example as a solid dosage form for oral administration. Co-administration generally refers to simultaneous or sequential administration such that therapeutically effective amounts of the compounds are both present in the body of the patient.

Co-administration includes administration of unit dosages of Compound A and Compound B, for example, administration of Compound A and Compound B within seconds, minutes, or hours of the administration of one another. For example, in some embodiments, a unit dose of one compound is administered first, followed within seconds or minutes by administration of a unit dose of other of the compounds.

In certain embodiments, Compound A and Compound B are administered orally.

In certain embodiments, Compound B and Compound A are formulated as a tablet. In certain embodiments, the tablet can contain another active ingredient for treating HIV. In certain embodiments, such tablets are suitable for once daily dosing.

In certain embodiments, Compound B and Compound A are formulated as an implant In certain embodiments, such implants are suitable for implantation every 3 months or, according to another embodiment, every 6 months. Examples of implant that may incorporate the combination of the invention are, for instance, US2012/0277690, US2004/0082937, or US2006/026475.

In another embodiment, the combination of Compound A and Compound B is administered to the patient once a day.

In another embodiment, the combination of Compound A and Compound B is administered to the patient twice a day.

In practice, the amount of each compound to be administered ranges from about 0.001 to 100 mg per kg of body weight, such total dose being given at one time or in divided doses. Each compound will be administered as a formulation in association with one or more pharmaceutically acceptable excipients. Alternatively, both compounds will be combined and administered as a formulation in association with one or more pharmaceutically acceptable excipients. The choice of excipient will, to a large extent, depend u p on factors such as the particular mode of administration, the effect of the excipient on solubility and stability, and the nature of the dosage form. Such compositions and methods for their preparation may be found, for example, in REMINGTON'S PHARMACEUTICAL SCIENCES (19th Edition, Mack Publishing Company, 1995).

In another embodiment, Compound A is administered to the patient orally at about 25 mg, about 30 mg, about 35 mg, about 40 mg, about 45 mg dose, about 50 mg, about 55 mg, about 60 mg, about 65 mg, about 70 mg, about 75 mg, about 80 mg, about 85 mg, about 90 mg, about 95 mg or about 100 mg, once, twice or three times a day. In another embodiment, cabotegravir is administered to the patient orally at about 25 mg to 100 mg, at about 25 mg to 75 mg, at about 35 mg to 65 mg or about 45 mg to 55 mg, once or twice per day. In another embodiment, cabotegravir is administered to the patient at about 50 mg, once or twice per day.

In another embodiment, Compound A is administered to a patient orally at about 100 mg to about 500 mg once a week. In another embodiment, Compound A is administered at about 100 mg, 125 mg, 150 mg, 200 mg, 250 mg, 300 mg, 350 mg, 400 mg, 450 mg, or 500 mg weekly.

In another embodiment, Compound A is administered to the patient parenterally at about 100-1000 mg per dose, more preferably a dose of about 100 mg, 200 mg, 300 mg, 400 mg, 500 mg, 600 mg, 700 mg, 800 mg, 900 mg, or 1000 mg. The parenteral dose is preferably administered weekly, or once every 1 week, 2 weeks, 3 weeks, 4 weeks, 5 weeks, 6 weeks, 7 weeks, 8 weeks, 9 weeks, 10 weeks, 11 weeks, or 12 weeks. Preferably Compound A is administered parenterally every 4 weeks or every 8 weeks.

In another embodiment, Compound B is administered to the patient orally at about 0.1 to 100 mg. More preferably, Compound B is administered orally at a dose of 0.25 mg, 0.5 mg, 1 mg, 2 mg, 10 mg, or 30 mg. In another embodiment, Compound B is administered to the patient orally once or twice per day. In another embodiment, Compound B is administered to the patient at about 10 mg once a day.

In another embodiment, Compound B is administered to a patient orally at about 2 mg to about 20 mg once a week. In another embodiment, Compound B is administered at about 2 mg, 2.5 mg, 4.5 mg, 5 mg, 6.75 mg, 7.5 mg, 9 mg, 10 mg, 11.25 mg, 12.5 mg, 15 mg, 17.5 mg, 18 mg, or 20 mg weekly.

In another embodiment, Compound B is administered to the patient parenterally at about 100-1000 mg per dose, more preferably a dose of about 100 mg, 200 mg, 300 mg, 400 mg, 500 mg, 600 mg, 700 mg, 800 mg, 900 mg, or 1000 mg. The parenteral dose is preferably administered weekly, or once every 1 week, 2 weeks, 3 weeks, 4 weeks, 5 weeks, 6 weeks, 7 weeks, 8 weeks, 9 weeks, 10 weeks, 11 weeks, or 12 weeks. According to another embodiment, the parenteral dose is administered by way of implant every 1 month, 2 months, 3 months, 4 months, 5 months, or 6 months. Preferably Compound B is administered parenterally every 8 weeks. Preferably Compound B is administered as an implant every 6 months.

A method for the treatment or preventing of diseases, disorders, and conditions is provided herein. An example of a disease, disorder, or condition includes, but is not limited to, a retrovirus infection, or a disease, disorder, or condition associated with a retrovirus infection. Retroviruses are RNA viruses and are generally classified into the alpharetrovirus, betaretrovirus, deltaretrovirus, epsilonretrovirus, gammaretrovirus, lentivirus, and spumavirus families. Examples of retroviruses include, but are not limited to, human immunodeficiency virus (HIV).

The active agents of the disclosed combination therapy may be administered to a human in any conventional manner. While it is possible for the active agents to be administered as compounds, they are preferably administered as a pharmaceutical composition that can include contact with an acid or base, either in an ionic salt form or in contact with the base or acid (i.e., co-formers) without sharing ions. The salt, acid or base co-former, carrier, or diluent should be acceptable, in the sense of being compatible with the other ingredients and not deleterious to the recipient thereof. Examples of carriers or diluents for oral administration include, but are not limited to: cornstarch, lactose, magnesium stearate, talc, microcrystalline cellulose, stearic acid, povidone, crospovidone, dibasic calcium phosphate, sodium starch glycolate, hydroxypropyl cellulose (e.g., low substituted hydroxypropyl cellulose), hydroxypropylmethyl cellulose (e.g., hydroxypropylmethyl cellulose 2910), sodium lauryl sulfate, mannitol, sodium stearyl fumarate, and talc. Examples of salts and acid or base co-formers include fumarate, hemifumarate, sodium, and hydrochloride.

The pharmaceutical compositions may be prepared by any suitable method, such as those methods well known in the art of pharmacy, for example, methods such as those described in Gennaro, et al., REMINGTON'S PHARMACEUTICAL SCIENCES (18th ed., Mack Publishing Co., 1990), especially “Part 8: Pharmaceutical Preparations and their Manufacture”. Such methods include the step of bringing into association the compounds with the carrier or diluents and, optionally, one or more accessory ingredients. Such accessory ingredients include, but are not limited to: fillers, binders, excipients, disintegrants, lubricants, colorants, flavoring agents, sweeteners, preservatives (e.g., antimicrobial preservatives), suspending agents, thickening agents, emulsifying agents, and/or wetting agents.

In the following description of the examples, specific embodiments in which the invention may be practiced are described. These embodiments are described in sufficient detail to enable those skilled in the art to practice the invention. Other embodiments may be utilized and logical and other changes may be made without departing from the scope of the invention. The following detailed description is, therefore, not to be taken in a limiting sense, and the scope of the invention is defined only by the appended claims, along with the full scope of equivalents to which such claims are entitled.

In certain embodiments, the present disclosure provides a method for treating an HIV infection, comprising administering to a patient in need thereof a therapeutically effective amount of Compound A and Compound B, or a pharmaceutically acceptable composition thereof, in combination with a therapeutically effective amount of one or more additional therapeutic agents that are suitable for treating an HIV infection.

In one embodiment, pharmaceutical compositions comprising Compound A and Compound B, in combination with one or more (e.g., one, two, three, one or two, or one to three) additional therapeutic agents, and a pharmaceutically acceptable carrier, diluent or excipient are provided.

In one embodiment, kits comprising Compound A and Compound B, or a pharmaceutical salt thereof, in combination with one or more (e.g., one, two, three, one or two, or one to three) additional therapeutic agents are provided.

In certain embodiments, a method for treating or preventing an HIV infection in a human having or at risk of having the infection is provided, comprising administering to the human a therapeutically effective amount of Compound A and Compound B, in combination with a therapeutically effective amount of one or more (e.g., one, two, three, one or two, or one to three) additional therapeutic agents.

In the above embodiments, the additional therapeutic agent may be an anti-HIV agent. For example, in some embodiments, the additional therapeutic agent is chosen from: HIV protease inhibitors, HIV non-nucleoside or non-nucleotide inhibitors of reverse tran-scriptase, HIV nucleoside or nucleotide inhibitors of reverse transcriptase, HIV integrase inhibitors, HIV non-catalytic site (or allosteric) integrase inhibitors, HIV entry inhibitors (e.g., CCR5 inhibitors, gp41 inhibitors (i.e., fusion inhibitors) and CD4 attachment inhibitors), CXCR4 inhibitors, gp120 inhibitors, G6PD and NADH-oxidase inhibitors, HIV vaccines, HIV maturation inhibitors, latency reversing agents (e.g., histone deacetylase inhibitors, proteasome inhibitors, protein kinase C (PKC) activators, and BRD4 inhibitors), compounds that target the HIV capsid (“capsid inhibitors”; e.g., capsid polymerization inhibitors or capsid disrupting compounds, HIV nucleocapsid p7 (NCp7) inhibitors, HIV p24 capsid protein inhibitors), pharmacokinetic enhancers, immune-based therapies (e.g., Pd-1 modulators, Pd-L1 modulators, CTLA4 modulators, toll like receptors modulators, IL-15 agonists, HIV antibodies, bispecific antibodies and “antibody-like” therapeutic proteins (e.g., DARTs®, DUOBODIES®, BITES®, XmAbs®, TandAbs®, Fab derivatives) including those targeting HIV gp120 or gp41, combination drugs for HIV, HIV p 17 matrix protein inhibitors, IL-13 antagonists, Peptidylprolyl cis-trans isomerase A modulators, protein disulfide isomerase inhibitors, complement C5a receptor antagonists, DNA methyltransferase inhibitor, HIV vif gene modulators, Vif dimerization antagonists, HIV-1 viral infectivity factor inhibitors, TAT protein inhibitors, HIV-1 Nef modulators, Hck tyrosine kinase modulators, mixed lineage kinase-3 (MLK-3) inhibitors, HIV-1 splicing inhibitors, Rev protein inhibitors, integrin antagonists, nucleoprotein inhibitors, splicing factor modulators, COMM domain containing protein 1 modulators, HIV Ribo-nuclease H inhibitors, retrorocyclin modulators, CDK-9 inhibitors, dendritic ICAM-3 grabbing nonintegrin 1 inhibitors, HIV GAG protein inhibitors, HIV POL protein inhibitors, complement Factor H modulators, ubiquitin ligase inhibitors, d eoxycytidine kinase inhibitors, cyclin dependent kinase inhibitors proprotein convertase PC9 stimulators, ATP dependent RNA helicase DDX3X inhibitors, reverse transcriptase priming complex inhibitors, HIV gene therapy, PI3K inhibitors, compounds, such as those disclosed in WO 2013/006738 (Gilead Sciences), US 2013/0165489 (University of Pennsylvania), WO 2013/091096A1 (Boehringer Ingelheim), WO 2009/062285 (Boehringer Ingelheim), US20140221380 (Japan Tobacco), US 20140221378 (Japan Tobacco), WO 2010/130034 (Boehringer

Ingelheim), WO 2013/159064 (Gilead Sciences), WO 2012/145728 (Gilead Sciences), WO2012/003497 (Gilead Sciences), WO2014/100323 (Gilead Sciences), WO2012/145728 (Gilead Sciences), WO 2013/159064 (Gilead Sciences) and WO 2012/003498 (Gilead Sciences) and WO 2013/006792 (Pharma Resources), and other drugs for treating HIV, and combinations thereof.

In certain embodiments, the additional therapeutic chosen from: HIV protease inhibitors, HIV non-nucleoside or non-nucleotide inhibitors of reverse transcriptase, HIV nucleoside or nucleotide inhibitors of reverse transcriptase, HIV integrase inhibitors, HIV non-catalytic site (or allosteric) integrase inhibitors, pharmacokinetic enhancers, and combinations thereof.

In certain embodiments, Compound B and Compound A are formulated as a tablet that may optionally contain one or more other compounds useful for treating HIV. In certain embodiments, the tablet can contain another active ingredient for treating HIV, such as HIV protease inhibitors, HIV non-nucleoside or non-nucleotide inhibitors of reverse transcriptase, HIV nucleoside or nucleotide inhibitors of reverse transcriptase, HIV integrase inhibitors, HIV non-catalytic site (or allosteric) integrase inhibitors, pharmacokinetic enhancers, and combinations thereof. In certain embodiments, such tablets are suitable for once daily dosing.

In certain embodiments, the additional therapeutic agent may be chosen from one or more of:

(1) Combination drugs chosen from: ATRIPLA® (efavirenz+tenofovir disoproxil fumarate+emtricitabine), COMPLERA® (EVIPLERA®, rilpivirine+tenofovir disoproxil fumarate+emtricitabine), STRIBILD® (elvitegravir+cobicistat+tenofovir disoproxil fumarate+emtricitabine), lamivudine+nevirapine+zidovu dine, atazanavir sulfate+cobicistat, darunavir+cobicistat, efavirenz+lamivudine+tenofovir disoproxil fumarate, Vacc-4x+romidepsin, APH-0812, raltegravir+lamivudine, KALE-TRA® (ALUVIA lopinavir+ritonavir), atazanavir sulfate+ritonavir, COMBIVIR® (zidovudine+lamivudine, AZT+3TC), EPZICOM® (Livexa®, abacavir sulfate+lamivudine, ABC+3TC), TRIZIVIR® (abacavir sulfate+zidovudine+lamivudine, ABC+AZT+3TC), TRUVADA® (tenofovir disoproxil fumarate+emtricitabine, TDF+FTC), tenofovir+lamivudine, atazanavir+cobicistat, doravirine+lamivudine+tenofovir disoproxil fumarate, doravirine+lamivudine+tenofovir disoproxil and lamivudine+tenofovir disoproxil fumarate;
(2) HIV protease inhibitors chosen from: amprenavir, atazanavir, fosamprenavir, fosam-prenavir calcium, indinavir, indinavir sulfate, lopinavir, ritonavir, nelfinavir, nelfinavir mesylate, saquinavir, saquinavir mesylate, tipranavir, brecanavir, darunavir, DG-17, TMB-657 (PPL-100), TMC-310911, and TMB-657;
(3) HIV non-nucleoside or non-nucleotide inhibitors of reverse transcriptase chosen from: delavirdine, delavirdine mesylate, nevirapine, (+), etravirine, dapivirine, doravirine, efavirenz, KM023, VM-1500, lentinan, AIC-292 and KM-023;
(4) HIV nucleoside or nucleotide inhibitors of reverse transcriptase chosen from: VIDEX® and VIDEX® EC (didanosine, ddl), zidovudine, emtricitabine, didanosine, stavudine, zalcitabine, lamivudine, censavudine, abacavir, abacavir sulfate, amdoxovir, elvucitabine, alovudine, phosphazid, fozivudine tidoxil, apricitabine, amdoxovir, KP-1461, fosalvudine tidoxil, tenofovir, tenofovir disoproxil, tenofovir disoproxil fumarate, tenofovir disoproxil hemifumarate, adefovir, adefovir dipivoxil, and festinavir. (5) HIV integrase inhibitors chosen from: curcumin, derivatives of curcumin, chicoric acid, derivatives of chicoric acid, 3,5-dicaffeoylquinic acid, derivatives of 3,5-dicaffeoylquinic acid, aurintricarboxylic acid, derivatives of aurintricarboxylic acid, caffeic acid phen-ethyl ester, derivatives of caffeic acid phenethyl ester, tyrphostin, derivatives of tyrphostin, quercetin, derivatives of quercetin, raltegravir, MK-4250, and TIVICAY® (dolutegravir);
(6) HIV non-catalytic site, or allosteric, integrase inhibitors (NCINI) chosen from: CX-05168, CX-05045 and CX-14442;
(7) HIV gp41 inhibitors chosen from: enfuvirtide, sifuvirtide and albuvirtide;
(8) HIV entry inhibitors, such as cenicriviroc;
(9) HIV gp120 inhibitors chosen from: Radha-108 (Receptol) and BMS-663068;
(10) CCRS inhibitors chosen from: aplaviroc, vicriviroc, maraviroc, cenicriviroc, PRO-140, Adaptavir (RAP-101), nifeviroc (TD-0232), TD-0680, TBR-220 (TAK-220) and vMIP (Haimipu);
(11) CD4 attachment inhibitors, such as ibalizumab;
(12) CXCR4 inhibitors chosen from: plerixafor, ALT-1188, vMIP and Haimipu;
(13) Pharmacokinetic enhancers chosen from: cobicistat and ritonavir;
(14) Immune-based therapies chosen from: dermaVir, interleukin-7, lexgenleucel-T (VRX-496), plaquenil (hydroxychloroquine), proleukin (aldesleukin, IL-2), interferon alfa, interferonalfa-2b, interferon alfa-n3, pegylated interferon alfa, interferon gamma, hydroxyurea, mycophenolate mofetil (MPA) and its ester derivative mycophenolate mofetil (MMF), WF-10, ribavirin, IL-2, IL-2 XL, IL-12, polymer polyethyleneimine (PEI), Gepon, VGV-1, MOR-22, toll-like receptors modulators (tlr1, tlr2, tlr3, tlr4, tlr5, tlr6, tlr7, tlr8, tlr9, tlr10, tlr11, tlr12 and tlr13), BMS-936559, rintatolimod and IR-103;
(15) HIV vaccines chosen from: peptide vaccines, recombinant subunit protein vaccines, live vector vaccines, DNA vaccines, virus-like particle vaccines (pseudovirion vaccine), CD4-derived peptide vaccines, vaccine combinations, rgp120 (AIDSVAX), ALVAC HIV (vCP1521)/AIDSVAX B/E (gp120) (RV144), Remune, ITV-1, Cantre Vir,Ad5-ENVA-48,DCVax-001 (CDX-2401), PEP-6409, Vacc-4x, Vacc-C5, VAC-3S, multiclade DNA recombinant adenovirus-5 (rAdS), Pennvax-G, YRC-HIV MAB060-00-AB, AVX-101, Tat Oyi vaccine, AVX-201, HIV-LAMP-vax, Ad35, Ad35-GRIN, NAcGM3NSSP ISA-51, poly-ICLC adjuvanted vaccines, Tatlmmune, GTU-multi-HIV (FIT-06), AGS-004, gp140[delta]V2.TVI+MF-59, rVSVIN HIV-1 gag vaccine, SeV-Gag vaccine, AT-20, DNK-4, Ad35-GRIN/ENV, TBC-M4, HIVAX, HIVAX-2, NYVAC-HIV-PT1, NYVAC-HIV-PT4, DNA-HIV-PT123, VIChREPOL®, rAAV1-PG9DP, GOVX-B11, GOVX-B21, ThV-01, TUTI-16, VGX-3300, TVI-HIV-1, Ad-4 (Ad4-env Clade C+Ad4-mGag), EN41-FPA2, PreVaxTat, TL-01, SAV-001, AE-H, MYM-V1O1, CombiHIVvac, ADVAX, MYM-V201, monomeric gp120 HIV-1 subtype C vaccine (Novartis), MVA-CMDR, MVATG-17401, ETV-01, CDX-1401, rcAd26.MOS1.HIV-Env, and DNA-Ad5 gag/pol/nef/nev (HVTN505);
(16) HIV antibodies, bispecific antibodies and “antibody-like” therapeutic proteins (such as DARTs®, Duo-bodies®, Bites®, XmAbs®, TandAbs®, Fab derivatives), including BMS-936559, TMB-360 and those targeting HIV gp120 or gp41 are chosen from: bavituximab, UB-421, C2F5, C2G12, C4E10, C2F5+C2G12+C4E10, 3-BNC-117, KD-247, PGT145, PGT121, MDXO1O (ipilimumab), VRCO1, A32, 7B2, 10E8, VRC-07-523 and VRC07;
(17) latency reversing agents chosen from: histone deacetylase inhibitors such as Romidepsin, vorinostat, panobinostat; proteasome inhibitors such as VELCADE®; protein kinase C (PKC) activators such as Indolactam, prostratin, ingenol B and DAG-lactones, lono-mycin, GSK-343, PMA, SAHA, BRD4 inhibitors, IL-15, JQ1, amphotericin B, and disulfram;
(18) HIV nucleocapsid p7 (NCp7) inhibitors, such as azodicarbonamide;
(19) HIV maturation inhibitors chosen from: BMS-955176 and GSK-2838232;
(20) P13K inhibitors chosen from: idelalisib, AZD-8186, buparlisib, CLR-457, pictilisib, neratinib, rigosertib, rigosertib sodium, EN-3342, TGR-1202, alpelisib, duvelisib, UCB-5857, taselisib, XL-765, gedatolisib, VS-5584, copanlisib, CAI orotate, perifosine, RG-7666, GSK-2636771, DS-7423, panulisib, GSK-2269557, GSK-2126458, CUDC-907, PQR-309, INCB-040093, pilaralisib, BAY-1082439, puquitinib mesylate, SAR-245409, AMG-319, RP-6530, ZSTK-474, MLN-1117, SF-1126, RV-1729, sonolisib, LY-3023414, SAR-260301 and CLR-1401;
(21) the compounds disclosed in WO 2004/096286 (Gilead Sciences), WO 2006/110157 (Gilead Sciences), WO 2006/015261 (Gilead Sciences), WO 2013/006738 (Gilead Sciences), US 2013/0165489 (University of Pennsylvania), US20140221380 (Japan Tobacco), US20140221378 (Japan Tobacco), WO 2013/006792 (Pharma Resources), WO 2009/062285 (Boehringer Ingelheim), WO 2010/130034 (Boehringer Ingelheim), WO 2013/091096A1 (Boehringer Ingelheim), WO 2013/159064 (Gilead Sciences), WO 2012/145728 (Gilead Sciences), WO2012/003497 (Gilead Sciences), WO2014/100323 (Gilead Sciences), WO02012/145728 (Gilead Sciences), WO2013/159064 (Gilead Sciences) and WO 2012/003498 (Gilead Sciences); and
(22) other drugs for treating HIV chosen from: REP 9, cytolin, CYT-107, alisporivir, BanLec, MK-8507, AG-1105, TR-452, MK-8591, REP 9, NOV-205, IND-02, metenkefalin, PGN-007, Acemannan, Gamimune, SCY-635, prolastin, 1,5-dicaffeoylquinic acid, BIT-225, RPI-MN, VSSP, Hlviral, IM0-3100, SB-728-T, RPI-MN, VIR-576, HGTV-43, MK-1376, rHIV7-shl-TAR-CCR5RZ, MazF gene therapy, BlockAide, ABX-464, SCY-635, naltrexone, AAV-eCD4-Ig gene therapy, TEV-90110, TEV-90112, deferiprone, and PA-1050040 (PA-040).

### EXAMPLES

In one example of combination tablet, a formulation of the instant invention consists of:

| Ingredient | Amount (mg) |
|---|---|
| Cabotegravir Na | 30 |
| EFdA | 10 |
| Microcrystalline cellulose | 30 |
| Lactose | 75 |
| Povidone | 5.2 |
| Polysorbate 20 | 0.8 |
| Magnesium Stearate | 5.0 |

In another exemplary formulation, a parenteral formulation of the instant invention consists of:

| Ingredient | Amount (mg) |
|---|---|
| Cabotegravir Na | 300 |
| EFdA | 100 |
| Manitol | 75 |
| Polysorbate 20 | 40 |
| Polyethylene Glycol | 40 |
| Water | QS |

What is claimed is:

1. A combination comprising:
(i) a compound of formula (I) 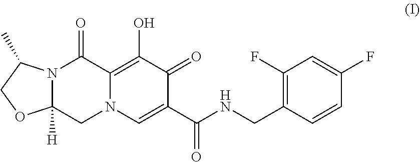 [SMILES: C[C@H]1CO[C@@H]2Cn3cc(C(=O)NCc4ccc(F)cc4F)c(=O)c(O)c3C(=O)N12]
or a pharmaceutically acceptable salt thereof; and
(ii) a compound of formula (II) 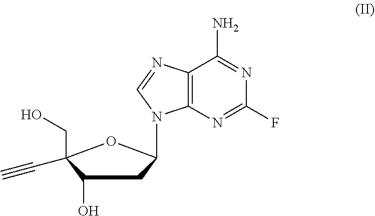 [SMILES: C#C[C@]1(CO)O[C@@H](n2cnc3c(N)nc(F)nc32)C[C@H]1O]
or a pharmaceutically acceptable salt thereof

2. A combination according to claim 1, wherein compound (i) is in the form of the sodium salt.

3. Use of a combination according to claim 1 in the manufacture of a medicament for the treatment of HIV.

4. A combination according to claim 1 for use in therapy.

5. A combination according to claim 4 for use in treating HIV.

6. A pharmaceutical composition comprising a combination according to claim 1 together with a pharmaceutically acceptable diluent or carrier.

7. A method of treating HIV in a human in need thereof which comprises the administration of a therapeutically effective amount of
(i) a compound of formula (I) 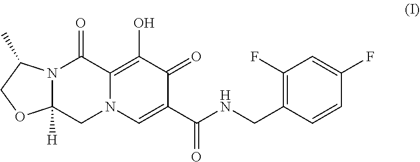 [SMILES: C[C@H]1CO[C@@H]2Cn3cc(C(=O)NCc4ccc(F)cc4F)c(=O)c(O)c3C(=O)N12]
or a pharmaceutically acceptable salt thereof; and
(ii) a compound of formula (II) 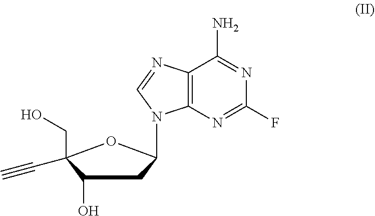 [SMILES: C#C[C@]1(CO)O[C@@H](n2cnc3c(N)nc(F)nc32)C[C@H]1O]
or a pharmaceutically acceptable salt thereof for use in therapy.

8. The method of claim 7, wherein compound (i) is in the form of the sodium salt.

9. The method of claim 7, wherein at least one of compounds (i) or (ii) is administered orally.

10. The method of claim 7, wherein at least one of the compounds (i) or (ii) is administered parenterally.

11. The method of claim 7, wherein the compounds are administered no more frequently than once every 4 weeks.

12. The method of claim 7, wherein the compounds are administered no more frequently than once every 8 weeks.

13. The method of claim 7 wherein the compounds are co-administered in a single dosage form.
